(19) United States
(12) Patent Application Publication
(10) Pub. No.: US 2014/0030241 A1
(43) Pub. Date: Jan. 30, 2014

(54) NUTRITIONAL COMPOSITIONS FOR INCREASING ARGININE LEVELS AND METHODS OF USING SAME

(71) Applicant: Norman Alan Greenberg, New Hope, MN (US)

(72) Inventor: Norman Alan Greenberg, New Hope, MN (US)

(73) Assignee: NESTEC S.A., Vevey (CH)

(21) Appl. No.: 14/008,929

(22) PCT Filed: Mar. 15, 2012
(86) PCT No.: PCT/EP12/54580

§ 371 Date: Sep. 30, 2013

Related U.S. Application Data
(60) Provisional application No. 61/470,447, filed on Mar. 31, 2011.
(60) Provisional application No. 61/490,037, filed on May 25, 2011.

(51) Int. Cl. A61K 31/711; A23L 1/29; A61K 31/198; A61K 31/7105; A61K 31/202

(52) U.S. Cl. CPC A61K 31/711; A61K 31/7105; A61K 31/202; A61K 31/198; A23L 1/296
USPC 424/93.41; 514/44 R; 514/5.5; 514/5.7; 514/5.6; 424/93.4; 424/93.48; 424/93.45; 424/93.42; 424/93.44; 424/657; 424/682; 424/655; 424/638; 424/667; 424/646; 424/639; 424/601; 424/722; 424/702; 424/600; 424/650; 424/643

(57) ABSTRACT

Nutritional compositions having dietary nucleotides, ω-3 fatty acids and citrulline are provided. The nutritional compositions may be formulated to improve T cell function, increase arginine availability in vivo, regulate myeloid-derived suppressor cells (“MDSC”), and decrease the risk and/or severity of infection after surgery or trauma. Methods of making, using and administering such nutritional compositions to individuals in need of same are also provided. Methods for modulating the affects of MDSC's are also provided.

### BACKGROUND

The present disclosure relates generally to health and nutrition. More specifically, the present disclosure relates to nutritional compositions having citrulline, nucleotides, and a source of ω-3 fatty acids. Methods of making and using the nutritional compositions are also provided. Methods of modulating myeloid-derived suppressor cells are also provided.

There are many types of nutritional compositions currently on the market. Nutritional compositions can be targeted toward certain consumer types, for example, young, elderly, athletic, etc., based on the specific ingredients of the nutritional composition. For example, it is important that individuals having undergone surgery and/or other trauma are provided a diet including nutritional compositions that promote proper healing. However, this is not always easy to accomplish because amounts of certain beneficial compounds in the body may naturally decrease in response to the trauma. Additionally, the body metabolizes different compositions in different ways and, as a result, may not be able to sufficiently counteract the body's natural depletion of such beneficial compounds.

One goal of nutritional support, therefore, is to provide individuals having undergone surgery and/or other trauma nutritional compositions that promote proper healing and decrease the risk and severity of infection. Another goal of nutritional support is to modulate the affects of myeloid-derived suppressor cells after surgery and/or other trauma to the body.

### SUMMARY

In the present disclosure, nutritional compositions are provided. The nutritional compositions may include citrulline in an amount from about 2 g/L to about 5.5 g/L, at least one nucleotide and a source of ω-3 fatty acids. The citrulline may also be present in an amount from about 2.5 g/L to about 4 g/L.

In an embodiment, the source of ω-3 fatty acids is selected from the group consisting of fish oil, krill, plant sources containing ω-3 fatty acids, flaxseed, walnut, algae, or combinations thereof. The ω-3 fatty acids may be selected from the group consisting of α-linolenic acid (“ALA”), docosahexaenoic acid (“DHA”), stearidonic acid (“SDA”), eicosapentaenoic acid (“EPA”), or combinations thereof. The source of ω-3 fatty acids may be present in an amount to provide the nutritional composition with about 1 to about 4 g ω-3 fatty acid/L, or with about 3 g ω-3 fatty acid/L.

In an embodiment, the at least one nucleotide is selected from the group consisting of a subunit of deoxyribonucleic acid (“DNA”), a subunit of ribonucleic acid (“RNA”), polymeric forms of DNA and RNA, yeast RNA, or combinations thereof. The at least one nucleotide may be an exogenous nucleotide and may be present in an amount from about 0.5 to about 3.0 g/L.

In an embodiment, the nutritional compositions include a phytonutrient selected from the group consisting of flavanoids, allied phenolic compounds, polyphenolic compounds, terpenoids, alkaloids, sulphur-containing compounds, or combinations thereof. The phytonutrient may be selected from the group consisting of carotenoids, plant sterols, quercetin, curcumin, limonin, or combinations thereof.

In an embodiment, the nutritional compositions include a source of protein. The source of protein may be present in an amount from about 15% to about 50% kcal. The source of protein may be selected from the group consisting of dairy based proteins, plant based proteins, animal based proteins, artificial proteins, or combinations thereof. The dairy based proteins may be selected from the group consisting of casein, caseinates, casein hydrolysate, whey, whey hydrolysates, whey concentrates, whey isolates, milk protein concentrate, milk protein isolate, or combinations thereof. The plant based proteins may be selected from the group consisting of soy protein, pea protein, wheat and fractionated wheat proteins, corn proteins, zein proteins, rice proteins, canola proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions include a source of protein. The source of protein may be present in an amount from about 15% to about 40% kcal. The source of protein may be selected from the group consisting of dairy based proteins, plant based proteins, animal based proteins, artificial proteins, or combinations thereof. The dairy based proteins may be selected from the group consisting of casein, caseinates, casein hydrolysate, whey, whey hydrolysates, whey concentrates, whey isolates, milk protein concentrate, milk protein isolate, or combinations thereof. The plant based proteins may be selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions include a source of protein. The source of protein may be present in an amount from about 15% to about 30% kcal. The source of protein may be selected from the group consisting of dairy based proteins, plant based proteins, animal based proteins, artificial proteins, or combinations thereof. The dairy based proteins may be selected from the group consisting of casein, caseinates, casein hydrolysate, whey, whey hydrolysates, whey concentrates, whey isolates, milk protein concentrate, milk protein isolate, or combinations thereof. The plant based proteins may be selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, or combinations thereof.

In an embodiment, the nutritional compositions include a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the nutritional compositions include a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

In an embodiment, the nutritional compositions include an additional amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine, or combinations thereof.

In an embodiment, the nutritional compositions include an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the nutritional compositions include a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the nutritional compositions include a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof.

In another embodiment, methods of making a nutritional composition are provided. The methods include providing citrulline in an amount from about 2 g/L to about 5.5 g/L, at least one nucleotide and a source of ω-3 fatty acids, and mixing the citrulline, at least one nucleotide and a source of ω-3 fatty acids to form a nutritional composition. The citrulline may also be present in an amount from about 2.5 g/L to about 4 g/L.

In yet another embodiment, methods of modulating the arginine-depleting effects of myeloid-derived suppressor cells in an individual in need of same are provided. The methods include providing a nutritional composition comprising an effective amount of citrulline, at least one nucleotide and a source of ω-3 fatty acids, and administering the nutritional composition to the individual. In an embodiment, the citrulline may be present in a supraphysiologic amount. The citrulline may also be present in an amount from about 2 g/L to about 5.5 g/L, or from about 2.5 g/L to about 4 g/L.

In still yet another embodiment, methods of modulating the arginine-depleting effects of myeloid-derived suppressor cells in an individual in need of same are provided. The methods include providing a nutritional composition comprising an effective amount of arginine, at least one nucleotide and a source of ω-3 fatty acids, and administering the nutritional composition to the individual. In an embodiment, the arginine may be present in a supraphysiologic amount. The arginine may also be present in an amount from about 8 g/L to about 24 g/L, or from about 12 g/L to about 18 g/L.

In yet another embodiment, methods of reducing the risk of infection in an individual that has recently experienced surgery and/or a trauma are provided. The methods include providing a nutritional composition comprising an effective amount of citrulline, at least one nucleotide and a source of ω-3 fatty acids, and administering the nutritional composition to the individual. In an embodiment, the citrulline may be present in a supraphysiologic amount. The citrulline may also be present in an amount from about 2 g/L to about 5.5 g/L, or from about 2.5 g/L to about 4 g/L.

In yet another embodiment, methods of reducing the risk of infection in an individual that has recently experienced surgery and/or a trauma are provided. The methods include providing a nutritional composition comprising an effective amount of arginine, at least one nucleotide and a source of ω-3 fatty acids, and administering the nutritional composition to the individual. In an embodiment, the arginine may be present in a supraphysiologic amount. The arginine may also be present in an amount from about 8 g/L to about 24 g/L, or from about 12g/L to about 18 g/L.

In still yet another embodiment, methods for improving the function of T lymphocytes in an individual in need of same are provided. The methods include providing a nutritional composition comprising an effective amount of citrulline, at least one nucleotide and a source of ω-3 fatty acids, and administering the nutritional composition to the individual. In an embodiment, the citrulline may be present in a supraphysiologic amount. The citrulline may also be present in an amount from about 2 g/L to about 5.5 g/L, or from about 2.5 g/L to about 4 g/L.

In another embodiment, methods for improving the function of T lymphocytes in an individual in need of same are provided. The methods include providing a nutritional composition comprising an effective amount of arginine, at least one nucleotide and a source of ω-3 fatty acids, and administering the nutritional composition to the individual. In an embodiment, the arginine may be present in a supraphysiologic amount. The arginine may also be present in an amount from about 8 g/L to about 24 g/L, or from about 12g/L to about 18 g/L.

In an embodiment, the individual has experienced a trauma selected from the group consisting of abrasions, contusions, lacerations, punctures, avulsions, amputations, eviscerations, burns, surgical trauma, or combinations thereof.

An advantage of the present disclosure is to provide improved nutritional compositions.

Another advantage of the present disclosure is to provide nutritional compositions that increase arginine levels in vivo.

Yet another advantage of the present disclosure is to provide nutritional compositions that reduce the arginine depleting effects of myeloid-derived suppressor cells.

Still yet another advantage of the present disclosure is to provide nutritional compositions that improve T cell function.

Another advantage of the present disclosure is to provide nutritional compositions that reduce the risk of infection after surgery or trauma.

Yet another advantage of the present disclosure is to provide nutritional compositions that decrease the severity of infection after surgery or trauma.

Additional features and advantages are described herein, and will be apparent from the following Detailed Description.

### DETAILED DESCRIPTION

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, sources of ω-3 fatty acids include, for example, fish oil, krill, plant sources of ω-3, flaxseed, walnut, and algae. Examples of ω-3 fatty acids include, for example, α-linolenic acid (“ALA”), docosahexaenoic acid (“DHA”), stearidonic acid (“SDA”), eicosapentaenoic acid (“EPA”), or combinations thereof

As used herein, “food grade micro-organisms” means micro- organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., Modern food microbiology, 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (‘non replicating’ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and ω-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., Probiotics: how should they be defined?, Trends Food Sci. Technol. 1999:10, 107-10. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

As used herein, a “processed whole food” is a whole food that has been modified from its natural or prepared state and is in a state so that it can be placed into a tube feed formulation.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, caseinates, casein hydrolysate, whey, whey hydrolysates, whey concentrates, whey isolates, milk protein concentrate, milk protein isolate, or combinations thereof. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof

All dosage ranges contained within this application are intended to include all numbers, whole or fractions, contained within said range.

As used herein, a “synbiotic” is a supplement that contains both a prebiotic and a probiotic that work together to improve the microflora of the intestine.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

The present disclosure is related to nutritional compositions that are formulated to increase T cell function, increase nitric oxide production and decrease the risk and severity of infection after surgery and/or trauma. The present disclosure is also related to methods of making and using same. In a general embodiment, the nutritional compositions include a source of ω-3 fatty acids, nucleotides, and citrulline. The methods include administering to an individual nutritional compositions including a source of ω-3 fatty acids, nucleotides, and an amino acid selected from the group consisting of arginine, citrulline, or combinations thereof. With respect to the present disclosure, Applicant has surprisingly found that compositions having a source of ω-3 fatty acids, nucleotides, and an amino acid selected from the group consisting of arginine, citrulline, or combinations thereof, provide a synergistic effect upon ingestion that is capable of modulating the arginine-depleting affects of myeloid derived suppressor cells after surgery and/or other trauma.

Arginine deficiency commonly develops after surgery and/or other trauma as a result of physiological changes to the insult inflicted on the body. Arginine deficiency is thought to be a result of increased destruction of arginine by myeloid- derived suppressor cells (“MDSC”) expressing arginase 1. MDSCs are a heterogeneous population of early myeloid progenitors, immature granulocytes, macrophages, and dendritic cells at different stages of differentiation. These cells have the capacity to suppress both the cytotoxic activities of natural killer (“NK”) and NKT cells, and the adaptive immune response mediated by C4⁺ and CD8⁺ T cells. MDSCs are induced by pro-inflammatory cytokines and are found in increased numbers in infectious and inflammatory pathological conditions. They can accumulate in the blood, bone marrow, and secondary lymphoid organs, can expand during cancer, inflammation and infection, and have a remarkable ability to suppress T cell responses. These cells constitute a unique component of the immune system that regulates immune responses in healthy individuals and in the context of various diseases.

After surgery and/or other trauma, arginine deficiency can cause T cell (T lymphocyte) dysfunction and decrease nitric oxide production, which, in turn, increases the risk of infection. If infection occurs, the severity of the infection can also be dramatically increased. Applicant has surprisingly found, however, that administration of a nutritional composition having an amino acid such as arginine or citrulline, a source of ω-3 fatty acids, and at least one nucleotide to an individual having experienced trauma (e.g., surgery or other trauma) improves T cell function and decreases the risk of infection after trauma. Although not wishing to be bound by any theories, Applicant believes that dietary nucleotides help to increase arginine availability through regulation of arginase 1 expression and/or regulation of myeloid-derived suppressor cells. Through this mechanism, the nutritional compositions and methods of the present disclosure are able to overcome arginine deficiencies observed after surgery/trauma, modulate the effects of myeloid-derived suppressor cells, restore arginine homeostasis and, thus, improve T cell function. This improvement in T cell function decreases the risk and severity of infection after surgery/trauma.

Arginine has many effects in the body that include, among others, modulation of immune function, would healing, hormone secretion, vascular tone, insulin sensitivity, and endothelial function. Arginine is metabolized into citrulline and nitric oxide (“NO”) via the enzyme nitric oxide synthase (“NOS”). However, only a portion of the arginine consumed by an individual remains available for metabolization to NO. As much as 60% of ingested arginine is metabolized in the liver by arginase before entering the circulation, where any remaining arginine may be metabolized to citrulline and NO. Accordingly, the ingestion of a large quantity of an arginine-rich dietary supplement is required in order to provide an effective amount of arginine to an individual having experienced surgery or other trauma. This limits the usefulness of arginine for a proper immune response to trauma.

An alternative source for arginine is the endogenous production of arginine from the amino acid citrulline. This route contributes about 20% to whole body arginine production. Citrulline is a precursor to L-arginine and is produced in the intestine. Just as arginine is converted to citrulline and NO, L-citrulline is converted to arginine in the mitochondria via a part of the urea cycle. The majority of circulating L-citrulline is converted in the kidneys, which are compromised of highly metabolically active tissue. As such, L-citrulline circulating in the bloodstream is first converted to arginine and then in cells to citrulline and NO. Further, citrulline enters circulation without being metabolized by the liver, with almost complete conversion to arginine in the kidneys. Therefore, smaller amounts of citrulline are required to provide the body with effective amounts of arginine in vivo. Moreover, ingestion of citrulline, or a precursor of citrulline, therefore, is able to provide many of the same benefits as ingestion of arginine including, for example, modulation of immune function, would healing, hormone secretion, vascular tone, insulin sensitivity, and endothelial function, but with lesser amounts.

Significantly, the conversion of L-citrulline to arginine occurs continuously, as long as L-citrulline is circulating in the bloodstream. As a result, circulating L-citrulline makes it possible to maintain elevated concentrations of arginine over time, which in turn makes it possible to maintain a steady modulation of myeloid-derived suppressor cells. Accordingly, the administration of L-citrulline may be used to overcome arginine deficiencies observed after surgery/trauma, modulate the effects of myeloid-derived suppressor cells, restore arginine homeostasis and, thus, improve T cell function. This improvement in T cell function decreases the risk and severity of infection after surgery/trauma. Thus, the administration of citrulline in place of arginine could allow for the increased benefit of would healing.

The present nutritional compositions may be administered in one large bolus, or in several feedings per day. A full day of feeding for the nutritional compositions of the present disclosure may be from about 1000 kcal to about 2000 kcal. In an embodiment, a full day feeding of the present nutritional compositions is about 1500 kcal. As such, at 1.0 kcal/mL, the present nutritional compositions may be administered in an amount of about 1500 mL per day. The skilled artisan will appreciate, however, that the present nutritional compositions may be administered according to feeding regimens that are tailored to meet the specific needs of the individuals consuming the compositions. Moreover, a serving, or a serving size, as used herein, is about 8 ounces.

Citrulline may be provided in the nutritional compositions in an amount from about 1.0 to about 2.0 g per serving. In an embodiment, the nutritional composition is an oral nutritional supplement. The nutritional compositions may be administered in a manner so as to provide an individual with about 3 to about 8 g of citrulline per day. In an embodiment, the nutritional compositions may be administered in a manner so as to provide an individual with about 4 to about 6 g of citrulline per day.

The nutritional compositions may further include sources of ω-3 and/or ω-6 fatty acids. Examples of sources of ω-3 fatty acids include, for example, fish oil, krill, plant sources of ω-3, flaxseed, walnut, and algae. Non-limiting examples of ω-3 fatty acids include α-linolenic acid (“ALA”), docosahexaenoic acid (“DHA”), stearidonic acid (“SDA”), and eicosapentaenoic acid (“EPA”). Non-limiting examples of ω-6 fatty acids include linoleic acid (“LA”), arachidonic acid (“ARA”). A ratio of ω-6 to ω-3 fatty acids may be between about 1:1 and 2:1. In an embodiment, the ratio of ω-6 to ω-3 fatty acids is about 1.5:1.

The sources of ω-3 fatty acids should be provided in amounts sufficient to provide the nutritional compositions with ω-3 fatty acids in an amount from about 0.5 g to about 2 g per serving. In a low dose serving of, for example, a nutritional composition that is an oral nutritional supplement, the ω-3 fatty acids may be present in an amount of about 0.5 g. In a high dose serving of, for example, a nutritional composition that is an oral nutritional supplement, the ω-3 fatty acids may be present in an amount of about 1 g to about 1.5 g. The nutritional compositions can be administered to an individual in a manner so as to provide the individual with about 2 g to 5 g of ω-3 fatty acids per day. In an embodiment, the nutritional compositions are administered to an individual so as to provide the individual with about 3 g of ω-3 fatty acids per day.

The nutritional compositions of the present disclosure provide nucleotides. As a component of adenosine triphosphate and associated molecules, nucleotides are also necessary for energy metabolism. Demand for nucleotides is highest in tissues with rapid cell turnover such as the gut and immune cells. Nucleotides can be obtained through dietary intake and also through the salvage pathway. Endogenous synthesis of nucleotides, although a high energy requiring process, appears to be sufficient in healthy individuals. However, the need for exogenous (dietary source) nucleotides occurs during situations of growth or stress, e.g., gut injury, sepsis, immune challenge, surgery and/or other trauma. See, Kulkarni et al., “The Role of Dietary Sources of Nucleotides in Immune Function: A Review,” Journal of Nutrition, pp. 1442S-1446S (1994). Several segments of the population including, for example, the elderly, pediatric populations, sedentary, and those with wounds, may particularly benefit from exogenous nucleotides.

Although endogenous synthesis constitutes a major source of nucleotides, nucleotides can also be obtained in the form of nucleoproteins naturally present in all foods of animal and vegetable origin including, for example, animal protein, peas, yeast, beans and milk. Further, concentrations of RNA and DNA in foods are dependent on cell density. Thus, meat, fish and seeds have higher nucleotide content than milk, eggs and fruits. Consequently, organ meats, fresh seafood, and dried legumes are rich food sources.

Additionally, nucleotides can be beneficial in the nutritional management of surgery and/or trauma by improving the resistance to infection at the wound site. Chronic nucleotide supplementation may counteract the hormonal response associated with physiological stress, resulting in an enhanced immune response.

Extensive experimentation on the influence of dietary nucleotides on lymphocyte function and cellular immunity in rodent models has also been conducted. Evidence exists to assert that the absence of dietary nucleotides does significantly decrease specific and non-specific immune responses. Findings include decreased maturation and proliferation of lymphoid cells in response to mitogens, decreased resistance to bacterial and fungal infection, and increased allograft survival.

Lymphocyte differentiation and proliferation can be stimulated by specific nucleosides and, in turn, nucleotide metabolism may be influenced by stages of lymphocyte activation and function. Furthermore, de novo synthesis and salvage of purines and pyrimidines is increased in stimulated lymphocytes. In support, an established marker for undifferentiated T cells, terminal deoxynucleotidyl transferase (“TdT”), has been identified in undifferentiated bone marrow and thymocytes of rodents fed diets devoid of nucleotides.

In vitro and in vivo studies of rodents on nucleotide free diets have demonstrated suppressed cell-mediated immune responses. Splenic lymphocytes from nucleotide free hosts evidenced significant decreases in proliferate response to mitogens, decreased interleukin-2 (“IL-2”) production and lower levels of IL-2 receptor and Lyt-1 surface markers. IL-2 is a growth factor for lymphocytes, while Lyt-1 is a marker of helper-inducer T cell immunity. Delayed cutaneous hypersensitivity was also lower.

These responses were largely reversed with additions of RNA or uracil, suggesting a formidable role for pyrimidines and/or limited capacity for their salvage. Furthermore, dietary nucleotides were shown to reverse lost immune response secondary to protein-calorie malnutrition more so than calories and protein alone. However, this reversal was limited to pyrimidines.

Investigations of the role of nucleotides in bacterial and fungal infection have also revealed increased resistance. Rodents on nucleotide containing diets demonstrated significant resistance to intravenous challenge of Staphylococcus aureus compared to those on nucleotide free diets. A decreased ability to phygocytose S. aureus was observed. Moreover, decreased survival times were observed in rodents on a nucleotide free diet after similar challenge with Candida albicans. Additions of RNA or uracil, but not adenine were shown to increase survival time.

The immunosuppressive effects of nucleotide free diets have also produced prolonged cardiac allograft survival in rodents as well as synergistic immunosuppression with cyclosporine A. These findings evidence influence on T-helper cell numbers and function. Various mechanisms of action have been proposed to explain these findings. Restriction of exogenous nucleotides is believed to influence the initial phase of antigen processing and lymphocyte proliferation via action on the T-helper-inducer as evidenced by increased levels of TdT in primary lymphoid organs. This is also suggestive of suppression of uncommitted T-lymphocyte response. Also, nucleotide restriction may cause arrest of T lymphocytes in the G phase of the cell cycle, thus inhibiting transition of lymphocytes to the S phase to illicit necessary immunological signals. Nucleotide restriction may also lower the cytolytic activity of NK cells and lower macrophage activity.

Dietary or exogenous nucleotides may also modulate T-helper cell mediated antibody production. A review of studies investigating nucleotide actions on humoral immune response identified effects in in vitro and in vivo animal models as well as in vitro actions in human systems. In vitro findings in splenic rodent cells primed with T cell-dependent antigens displayed significant increases in the number of antibody producing cells in yeast RNA containing cultures. RNA additions to normal strains demonstrated similar results and were nullified by T cell depletion. Thus, the antibody did not increase in response to T cell independent antigens or polyclonal B cell activation. The specific antibody response of yeast RNA was attributed to nucleotides.

Immunoglobulin production has also been shown to increase in in vitro adult human peripheral blood mononuclear cell in response to T cell dependent antigen and stimuli. Specifically, this involved increased immunoglobulin M (“IgM”) and G (“IgG”) production. IgM production increased in the functionally immature umbilical cord mononuclear cells in response to T cell dependent stimuli as well.

Accordingly, in a state of nucleotide deficiency, incorporated dietary nucleotides could potentially exert similar immune effects in vivo. Antibody response to T cell dependent antigen was suppressed in rodents maintained on nucleotide free diets for prolonged periods, and immune function was rapidly restored with nucleotide supplementation. However, the mixture used for supplementation showed no effect on in vitro antibody production to antigen-dependent antigens suggestive of nucleotide effects on local, specific immune response. In addition, significant increases in the numbers of antigen-specific immunoglobulin-secreting cells were observed in rodent splenic cells in the presence of nucleotides. Additions of AMP, GMP or UMP have also resulted in increased IgG response in rodents. GMP was also shown to increase IgM response. Studies in preterm infants on nucleotide supplemented formulas have revealed increased circulating levels of IgM and IgA in the first three months of life as well as higher concentrations of specific IgG against α-casein and β-lactoglobulin in the first month of life. Specific IgG levels to low response antigens may also increase in normal infants receiving dietary nucleotide containing formulas.

Mechanistically, in vitro and in vivo observations are thought to involve nucleotide effects on T-helper-cells at antigen presentation, modulations via interactions with cell surface molecules of T cells, suppressed nonspecific activation of T cells in response to antigen stimulus, and increased specific antibody response mediated through resting T cells. Therefore, dietary nucleotides may favor the balance of T cell differentiation to T-helper-2-cells which are primarily involved in B-cell response. Thus, it is clear that nucleotides can present several physiological benefits to patients having any of the above-mentioned conditions including, for example, surgical trauma or other trauma.

The skilled artisan will appreciate that any known source of nucleotides may be used in the present nutritional compositions. For example, fruits and vegetables may be used in the present nutritional compositions, so long as the fruits and vegetables are a source of phytochemicals and/or nucleotides. Further, the skilled artisan will also appreciate that the fruits and/or vegetables may be provided in any amounts effective to provide the patient with a sufficient amount of phytochemicals and/or nucleotides to achieve the advantages described above. Although the known fruits and vegetables may provide a small amount of nucleotides, the primary benefit derived from nucleotides will be obtained by adding additional sources of exogenous nucleotides. In an embodiment, certain meats may serve as a source of exogenous nucleotides.

The skilled artisan will also appreciate that any effective amount of nucleotides may be used in the nutritional compositions. For example, dietary nucleotides may be present in an oral nutritional supplement in an amount of about 100 to about 800 mg per serving. In an embodiment, the nutritional compositions are administered to an individual in a manner so as to provide the individual with about 1.0 to about 2.5 g nucleotides per day. Amounts for a full day of feeding for the nutritional compositions of the present disclosure are as described above.

In an embodiment, the nutritional compositions include a source of phytochemicals. Phytochemicals are non-nutritive compounds that are found in many fruits and vegetables, among other foods. There are thousands of phytochemicals that can be categorized generally into three main groups. The first group is flavonoids and allied phenolic and polyphenolic compounds. The second group is terpenoids, e.g., carotenoids and plant sterols. The third group is alkaloids and sulfur containing compounds. Phytochemicals are active in the body and, in general, act similarly to antioxidants. They also appear to play beneficial roles in inflammatory processes, clot formation, asthma, and diabetes. Researchers have theorized that to receive the most benefit from consumption of phytochemicals, they should be consumed as part of whole foods, because of the complex, natural combination and potentially synergistic effects. This may partially explain the health benefits associated with consumption of whole fruits and vegetables. Increased intake of fruits and vegetables is associated with reduced risk of many chronic diseases. In order to enhance the phytochemical profile of the present nutritional compositions, in an embodiment, the compositions include various fruits and vegetables containing these compounds.

In an embodiment, the nutritional compositions include a source of protein. The protein source may be dietary protein including, but not limited to animal protein (such as milk protein, meat protein or egg protein), vegetable protein (such as soy protein, wheat protein, rice protein, canola protein, and pea protein), or combinations thereof. In an embodiment, the protein is selected from the group consisting of whey, chicken, corn, caseinate, wheat, flax, soy, canola, carob, pea or combinations thereof. In another embodiment, the protein is pea protein or pea protein isolate. In another embodiment, the protein is canola protein. The protein may be present in the nutritional compositions in an amount from about 15% to about 50% kcal, or from about 15% to about 40% kcal, or from about 15% to about 30% kcal or from about 20% to about 25% kcal. In an embodiment, protein is present in an amount of about 22% kcal.

In an embodiment, vegetable proteins will be included to further enhance the net alkaline profile of the formula and increase the variety of macronutrient sources. Based on the nutritional profile of specific vegetable proteins (e.g., pea protein isolate) there are limitations in the amount of vegetable protein sources that can be included in a formula. For example, the amino acid profile of pea protein includes all of the indispensable amino acids. Pea protein is relatively rich in arginine, but limiting in the sulphur-containing amino acids, methionine, and cysteine. However, it is possible, for example, to blend pea protein isolates with a complete protein source (such as milk protein or complete vegetable proteins) having sufficient sulphur-containing amino acids to offset such deficiency. Canola protein (i.e., isolates, hydrosylates and concentrates) is one such vegetable protein which can provide appreciable amounts of sulfur-containing amino acids to further augment the amino acid profile to deliver the necessary protein quality to the patient. Additionally, animal derived proteins are typically more abundant in sulphur-containing amino acids than vegetable proteins.

In an embodiment, the nutritional compositions of the present disclosure are lactose free and/or gluten free.

The nutritional compositions of the present disclosure may also include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof. The carbohydrates may be present in the nutritional compositions in an amount from about 30% to about 70% kcal, or from about 40% to about 50% kcal. In an embodiment, protein is present in an amount of about 50% kcal.

The nutritional compositions may also include grains. The grains may include, for example, whole grains, which may be obtained from different sources. The different sources may include semolina, cones, grits, flour and micronized grain (micronized flour), and may originate from a cereal or a pseudo-cereal. In an embodiment, the grain is a hydrolyzed whole grain component. As used herein, a “hydrolyzed whole grain component” is an enzymatically digested whole grain component or a whole grain component digested by using at least an alpha-amylase, which alpha-amylase shows no hydrolytic activity towards dietary fibers when in the active state. The hydrolyzed whole grain component may be further digested by the use of a protease, which protease shows no hydrolytic activity towards dietary fibers when in the active state. The hydrolyzed whole grain component may be provided in the form of a liquid, a concentrate, a powder, a juice, a puree, or combinations thereof.

A source of fat may also be included in the present nutritional compositions. The source of fat may include any suitable fat or fat mixture. For example, the fat source may include, but is not limited to, vegetable fat (such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

In an embodiment, the nutritional compositions further include one or more prebiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional compositions may further include one or more probiotics. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

One or more amino acids may also be present in the nutritional compositions. Non-limiting examples of amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine, or combinations thereof. In an embodiment, the amino acid is present in a supraphysiologic amount. In another embodiment, the amino acid is present in an amount from about 0.5 to about 10 g/L, or from about 1 to 8 g/L. In an embodiment, the amino acid is present in an amount from about 2 g/L to about 4 g/L.

One or more antioxidants may also be present in the nutritional compositions. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The nutritional compositions also include fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber. The source of fiber may be present in the nutritional compositions in an amount from about 5 to about 15 g/L. In an embodiment, the source of fiber is present in an amount of about 10 g/L.

The nutritional compositions of the present disclosure may be a source of either incomplete or complete nutrition. The nutritional compositions may be administered by oral administration or tube feeding. If the nutritional compositions are formulated to be administered orally, the compositions may be a liquid oral nutritional supplement or feeding. The nutritional compositions may also be used for short term or long term tube feeding.

In addition to the nutritional compositions comprising citrulline, at least one nucleotide and a source of ω-3 fatty acids, Applicant has also surprisingly found that nutritional compositions including the combination of arginine, at least one nucleotide and a source of ω-3 fatty acids aid in modulating the arginine-depleting affects of MDSC's. Indeed, as described above, arginine deficiency can cause T cell dysfunction and decrease nitric oxide production, which, in turn, increases the risk of infection. If infection occurs, the severity of the infection can also be dramatically increased. Applicant has surprisingly found, however, that administration of a nutritional composition having an amino acid such as arginine or citrulline, a source of ω-3fatty acids, and at least one nucleotide to an individual having experienced trauma (e.g., surgery or other trauma) improves T cell function and decreases the risk of infection after trauma. The trauma may include, for example, abrasions, contusions, lacerations, punctures, avulsions, amputations, eviscerations, burns, surgical trauma, or combinations thereof.

Although not wishing to be bound by any theories, Applicant believes that dietary nucleotides help to increase arginine availability through regulation of arginase 1 expression and/or regulation of myeloid-derived suppressor cells. Through this mechanism, the nutritional compositions and methods of the present disclosure are able to overcome arginine deficiencies observed after surgery/trauma, modulate the effects of myeloid-derived suppressor cells, restore arginine homeostasis and, thus, improve T cell function. This improvement in T cell function decreases the risk and severity of infection after surgery/trauma.

As such, the present disclosure also provides for methods for modulating the arginine-depleting effects of myeloid-derived suppressor cells in an individual in need of same are provided. The methods include providing a nutritional composition comprising an effective amount of citrulline or an effective amount of arginine, at least one nucleotide and a source of ω-3 fatty acids, and administering the nutritional composition to the individual. In an embodiment, the citrulline or arginine may be present in a supraphysiologic amount. In an embodiment wherein the nutritional composition includes citrulline, the citrulline may also be present in an amount from about 2 g/L to about 5.5 g/L, or from about 2.5 g/L to about 4 g/L. In an embodiment wherein the nutritional composition includes arginine, the arginine may be present in an amount from about 8 g/L to about 24 g/L. In an embodiment, the arginine may be present in an amount from about 12g/L to about 18 g/L. In an embodiment, the nutritional composition includes both citrulline and arginine.

Methods of reducing the risk of infection in an individual that has recently experienced surgery and/or a trauma are also provided. The methods include providing a nutritional composition comprising an effective amount of citrulline or an effective amount of arginine, at least one nucleotide and a source of ω-3 fatty acids, and administering the nutritional composition to the individual. In an embodiment, the citrulline or arginine may be present in a supraphysiologic amount. In an embodiment wherein the nutritional composition includes citrulline, the citrulline may also be present in an amount from about 2 g/L to about 5.5 g/L, or from about 2.5 g/L to about 4 g/L. In an embodiment wherein the nutritional composition includes arginine, the arginine may be present in an amount from about 8 g/L to about 24 g/L. In an embodiment, the arginine may be present in an amount from about 12 g/L to about 18 g/L. In an embodiment, the nutritional composition includes both citrulline and arginine.

Methods for improving the function of T lymphocytes in an individual in need of same are further provided. The methods include providing a nutritional composition comprising an effective amount of citrulline or an effective amount of arginine, at least one nucleotide and a source of ω-3 fatty acids, and administering the nutritional composition to the individual. In an embodiment, the citrulline or arginine may be present in a supraphysiologic amount. In an embodiment wherein the nutritional composition includes citrulline, the citrulline may also be present in an amount from about 2 g/L to about 5.5 g/L, or from about 2.5 g/L to about 4 g/L. In an embodiment wherein the nutritional composition includes arginine, the arginine may be present in an amount from about 8 g/L to about 24 g/L. In an embodiment, the arginine may be present in an amount from about 12 g/L to about 18 g/L. In an embodiment, the nutritional composition includes both citrulline and arginine.

By way of example and not limitation, the following Example is illustrative of advantages of nutritional compositions in accordance with the present disclosure.

### EXAMPLE

In an example, and to test the above-described nutritional compositions and advantages thereof, Applicant intends to perform tests on a group of experimental mice. In a control group, the mice will only receive the regular diet. In an experimental group, the diet will be enriched with nucleotides. One week after receiving the diet, the mice will be subjected to trauma by performing a laparotomy under general anesthesia. T lymphocyte function will be measured including T cell receptor zeta chain expression, T cell proliferation, cytotoxicity, production of interferon gamma, and memory response. MDSC activity and arginase will also be measured.

Applicant believes that the experiment will demonstrate that trauma will produce significant alterations in T cell function in the control group. In addition, it is believed that this group will exhibit a significant accumulation of MDSC and high arginase 1 expression. In contrast, it is believed that the experimental group will exhibit a significant “blunting” in the increase in MDSC and arginase 1 expression with preservation of T cell function.

Applicant also intends to perform additional experiments using combinations of arginine and nucleotides in the diet. Applicant believes that mice receiving both arginine and nucleotides will exhibit the best T cell responses.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising citrulline in an amount from about 2 g/L to about 5.5 g/L, at least one nucleotide and a source of ω-3 fatty acids.

2. The nutritional composition according to claim 1, wherein the citrulline is present in an amount from about 2.5 g/L to about 4 g/L.

3. The nutritional composition according to claim 1, wherein the source of ω-3 fatty acids is selected from the group consisting of fish oil, krill, plant sources containing ω-3 fatty acids, flaxseed, walnut, algae, and combinations thereof.

4. The nutritional composition according to claim 1, wherein the ω-3 fatty acids are selected from the group consisting of α-linolenic acid (“ALA”), docosahexaenoic acid (“DHA”), stearidonic acid (“SDA”), eicosapentaenoic acid (“EPA”), and combinations thereof.

5. The nutritional composition according to claim 1, wherein the source of ω-3 fatty acids is present in an amount sufficient to provide the nutritional composition with about 1 to about 4 g ω-3 fatty acid/L.

6. The nutritional composition according to claim 5, wherein the source of ω-3 fatty acids is present in an amount sufficient to provide the nutritional composition with about 3 g ω-3 fatty acid/L.

7. The nutritional composition according to claim 1, wherein the at least one nucleotide is selected from the group consisting of a subunit of deoxyribonucleic acid (“DNA”), a subunit of ribonucleic acid (“RNA”), polymeric forms of DNA and RNA, yeast RNA, and combinations thereof.

8. The nutritional composition according to claim 1, wherein the at least one nucleotide is an exogenous nucleotide.

9. The nutritional composition according to claim 1, wherein the at least one nucleotide is present in an amount from about 0.5 to about 3.0 g/L.

10. The nutritional composition according to claim 1 comprising a phytonutrient selected from the group consisting of flavanoids, allied phenolic compounds, polyphenolic compounds, terpenoids, alkaloids, sulphur-containing compounds, carotenoids, plant sterols, quercetin, curcumin, limonin, and combinations thereof.

11. The nutritional composition according to claim 1 including a source of protein.

12. The nutritional composition according to claim 11, wherein the source of protein is present in an amount from about 15% to about 50% kcal.

13. The nutritional composition according to claim 11, wherein the protein is selected from the group consisting of casein, caseinates, casein hydrolysate, whey, whey hydrolysates, whey concentrates, whey isolates, milk protein concentrate, milk protein isolate, and combinations thereof.

14. The nutritional composition according to claim 11, wherein the protein is selected from the group consisting of soy protein, pea protein, canola protein, wheat and fractionated wheat proteins, corn proteins, zein proteins, rice proteins, oat proteins, potato proteins, peanut proteins, green pea powder, green bean powder, spirulina, proteins derived from vegetables, beans, buckwheat, lentils, pulses, single cell proteins, and combinations thereof.

15. The nutritional composition according to claim 1 comprising a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides , resistant starches, retrograded starch, sialooligosaccharides, sialyllactose, soyoligosaccharides, sugar alcohols, xylooligosaccharides, their hydrolysates, and combinations thereof.

16. The nutritional composition according to claim 1 comprising a probiotic selected from the group consisting of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, and combinations thereof.

17. The nutritional composition according to claim 1 comprising an additional amino acid selected from the group consisting of alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, ornithine, and combinations thereof.

18. The nutritional composition according to claim 1 comprising an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof.

19. The nutritional composition according to claim 1 comprising a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof.

20. The nutritional composition according to claim 1 comprising a mineral selected from the group consisting of boron, calcium, chromium, copper, iodine, iron, magnesium, manganese, molybdenum, nickel, phosphorus, potassium, selenium, silicon, tin, vanadium, zinc, and combinations thereof.

21. A method of making a nutritional composition, the method comprising:
providing citrulline in an amount from about 2 g/L to about 5.5 g/L, at least one nucleotide and a source of ω-3 fatty acids; and
mixing the citrulline, at least one nucleotide and a source of ω-3 fatty acids to form a nutritional composition.

22. A method of modulating the arginine-depleting effects of myeloid-derived suppressor cells in an individual in need of same, the method comprising the steps of:
providing a nutritional composition comprising an effective amount of citrulline, at least one nucleotide and a source of ω-3 fatty acids; and
administering the nutritional composition to the individual.

23. The method according to claim 22, wherein the effective amount of citrulline is a supraphysiologic amount.

24. A method of modulating the arginine-depleting effects of myeloid-derived suppressor cells in an individual in need of same, the method comprising the steps of:
providing a nutritional composition comprising an effective amount of arginine, at least one nucleotide and a source of ω-3 fatty acids; and
administering the nutritional composition to the individual.

25. The method according to claim 24, wherein the effective amount of arginine is a supraphysiologic amount of arginine.

26. The method according to claim 24, wherein the effective amount of arginine is an amount from about 8 g/L to about 24 g/L.

27. A method of reducing the risk of infection in an individual that has recently experienced surgery and/or a trauma, the method comprising the steps of:
providing a nutritional composition comprising an effective amount of citrulline, at least one nucleotide and a source of ω-3 fatty acids; and
administering the nutritional composition to the individual.

28. The method according to claim 27, wherein the effective amount of citrulline is a supraphysiologic amount.

29. A method of reducing the risk of infection in an individual that has recently experienced surgery and/or a trauma, the method comprising the steps of:
providing a nutritional composition comprising an effective amount of arginine, at least one nucleotide and a source of ω-3 fatty acids; and
administering the nutritional composition to the individual.

30. The method according to claim 29, wherein the effective amount of arginine is a supraphysiologic amount of arginine.

31. The method according to claim 29, wherein the effective amount of arginine is an amount from about 8 g/L to about 24 g/L.

32. A method of improving the function of T lymphocytes in an individual in need of same, the method comprising the steps of:
providing a nutritional composition comprising an effective amount of citrulline, at least one nucleotide and a source of ω-3 fatty acids; and
administering the nutritional composition to the individual.

33. The method according to claim 32, wherein the individual has experienced a trauma selected from the group consisting of abrasions, contusions, lacerations, punctures, avulsions, amputations, eviscerations, burns, surgical trauma, and combinations thereof.

34. The method according to claim 32, wherein the effective amount of citrulline is a supraphysiologic amount.

35. A method of improving the function of T lymphocytes in an individual in need of same, the method comprising the steps of:
providing a nutritional composition comprising an effective amount of arginine, at least one nucleotide and a source of ω-3 fatty acids; and
administering the nutritional composition to the individual.

36. The method according to claim 35, wherein the individual has experienced a tissue trauma selected from the group consisting of abrasions, contusions, lacerations, punctures, avulsions, amputations, eviscerations, burns, surgical trauma, and combinations thereof.

37. The method according to claim 35, wherein the effective amount of arginine is a supraphysiologic amount of arginine.

38. The method according to claim 35, wherein the effective amount of arginine is an amount from about 8 g/L to about 24 g/L.
